(19) United States
(12) Patent Application Publication
(10) Pub. No.: US 2026/0000270 A1
(43) Pub. Date: Jan. 1, 2026

(54) IMAGE PROCESSING DEVICE, IMAGE PROCESSING METHOD, AND STORAGE MEDIUM

(71) Applicant: NEC Corporation, Minato-ku, Tokyo (JP)

(72) Inventors: Ryosaku SHINO, Tokyo (JP); Kenichi KAMIJO, Tokyo (JP); Tatsu KIMURA, Tokyo (JP)

(73) Assignee: NEC Corporation, Minato-ku, Tokyo (JP)

(21) Appl. No.: 18/881,403

(22) PCT Filed: Jul. 21, 2022
(86) PCT No.: PCT/JP2022/028321

§ 371 Date: Jan. 6, 2025

(51) Int. Cl. A61B 1/00; G06T 7/00

(52) U.S. Cl. CPC A61B 1/00055; A61B 1/000094; A61B 1/0005; G06T 7/0012; G06T 2207/10068; G06T 2207/30096

(57) ABSTRACT

The image processing device 1X includes an infiltration distance acquisition means 32X and an output control means 33X. The infiltration distance acquisition means 32X is configured to acquire, based on an endoscopic image obtained by photographing an examination target by a photographing unit provided in an endoscope, an infiltration distance of a tumor part of the examination target in the endoscopic image. The output control means 33X is configured to output an image or sound based on the infiltration distance to an output device. It can be used to support examiner's decision making and the like.

### TECHNICAL FIELD

The present disclosure relates to a technical field of an image processing device, an image processing method, and a storage medium for processing an image acquired in endoscopic examination.

### BACKGROUND

There is a conventional endoscopic examination system which displays an image of the lumen of an organ. For example, Patent Literature 1 discloses a support method of diagnosing a disease using an endoscopic image of a digestive organ.

### CITATION LIST

### Patent Literature

Patent Literature 1: JP 2020-078539A

### SUMMARY

### Problem to be Solved

The technique using CAD (Computer Aided Detection/Diagnosis) which supports detecting and diagnosing a lesion part from an image captured in the endoscopic examination has been proposed. On the other hand, when there is a tumor, the distribution of the degree of infiltration of the tumor has to be determined through detailed examination such as biopsy.

In view of the above-described issue, it is therefore an example object of the present disclosure to provide an image processing device, an image processing method, and a storage medium capable of presenting information on a tumor in endoscopic examination.

### Means for Solving the Problem

One mode of the image processing device is an image processing device including:
an infiltration distance acquisition means configured to acquire,
   based on an endoscopic image obtained by photographing an examination target by a photographing unit provided in an endoscope,
   an infiltration distance of a tumor part of the examination target in the endoscopic image; and
an output control means configured to output an image or sound based on the infiltration distance by an output device.

One mode of the image processing method is an image processing method executed by a computer, the image processing method including:
acquiring,
   based on an endoscopic image obtained by photographing an examination target by a photographing unit provided in an endoscope,
   an infiltration distance of a tumor part of the examination target in the endoscopic image; and
outputting an image or sound based on the infiltration distance by an output device.

One mode of the storage medium is a storage medium storing a program executed by a computer, the program causing the computer to:
acquire,
   based on an endoscopic image obtained by photographing an examination target by a photographing unit provided in an endoscope,
   an infiltration distance of a tumor part of the examination target in the endoscopic image; and
output an image or sound based on the infiltration distance by an output device.

### Effect

An example advantage according to the present invention is to present information on a tumor in endoscopic examination.

### BRIEF DESCRIPTION OF THE DRAWINGS

FIG. 1 It illustrates a schematic configuration of an endoscope examination system.

FIG. 2 It illustrates a hardware configuration of an image processing device.

FIGS. 3A to 3D They schematically illustrate a flow of display process based on the infiltration distance of a tumor part.

FIG. 4 It is a functional block diagram of an image processing device relating to display processing based on the infiltration distance of a tumor part.

FIGS. 5A and 5B FIG. 5A illustrates an outline of the estimation process of the infiltration distance based on the first example.

FIG. 5B is a diagram showing an outline of a method of estimating the infiltration distance along the cross section designation line.

FIG. 6 It illustrates the first display example of the display screen image displayed on the display device in the endoscopic examination.

FIG. 7 It illustrates the second display example of the display screen image displayed on the display device in the endoscopic examination.

FIG. 8 It is an example of a flowchart showing an outline of a process performed by the image processing device during the endoscopic examination in the first example embodiment.

FIG. 9 It is a schematic configuration diagram of an endoscopic examination system according to a modification.

FIG. 10 It is a block diagram of an image processing device according to the second example embodiment.

FIG. 11 It is an example of a flowchart executed by the image processing device in the second example embodiment.

### EXAMPLE EMBODIMENTS

Hereinafter, example embodiments of an image processing device, an image processing method, and a storage medium will be described with reference to the drawings.

### First Example Embodiment

### (1) System Configuration

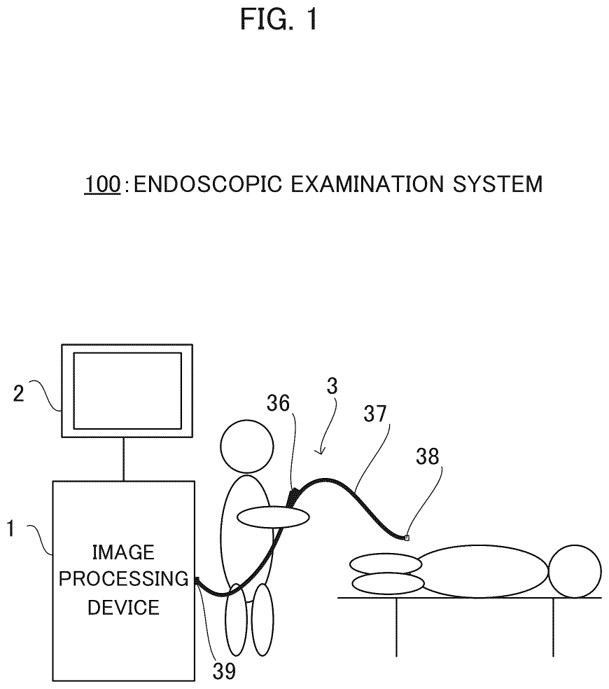

FIG. 1 shows a schematic configuration of an endoscopic examination system 100. As shown in FIG. 1, an endoscopic examination system 100 is a system for presenting information related to a part (also referred to as “tumor part”) which is suspected of a tumor in an examination target to an examiner such as a doctor who performs examination or treatment using an endoscope, and mainly includes an image processing device 1, a display device 2, and an endoscope 3 connected to the image processing device 1.

The image processing device 1 acquires a time series of images (also referred to as “endoscopic images Ia”) captured by the endoscope 3 from the endoscope 3 and displays a screen image based on the endoscopic images Ia on the display device 2. The endoscopic images Ia are images captured according to a predetermined frame cycle during at least one of the insertion process of the endoscope 3 to a subject and/or the ejection process of the endoscope 3 from the subject. In the present example embodiment, upon detecting an endoscopic image Ia (also referred to as “tumor-containing image”) which includes a tumor part, the image processing device 1 estimates the infiltration distance (i.e., the depth of the tumor) of the tumor part of the examination target in the tumor-containing image, and causes the display device 2 to display an image based on the estimated infiltration distance. As described below, examples of the “image based on the infiltration distance” includes a map indicating the infiltration distances, a cross section view at a cross section plane specified by the user, and a three-dimensional model representing a three-dimensional shape of the tumor by CG (Computer Graphics).

The display device 2 is a display or the like for displaying information based on the display signal supplied from the image processing device 1.

The endoscope 3 mainly includes an operation unit 36 for examiner to perform a predetermined input, a shaft 37 which has flexibility and which is inserted into the organ to be photographed of the subject, a tip unit 38 having a built-in photographing unit such as an ultra-small image pickup device, and a connecting unit 39 for connecting with the image processing device 1. In the present example embodiment, the operation unit 36 includes a button (also referred to as “still image saving button”) for instructing capture (i.e., saving as a still image) of an endoscopic image displayed on the display device 2 when the examiner determines that the endoscopic image including a tumor part is displayed on the display device 2.

The configuration of the endoscope examination system 100 shown in FIG. 1 is an example, and various change may be applied to the configuration. For example, the image processing device 1 may be configured integrally with the display device 2. In another example, the image processing device 1 may be configured by a plurality of devices.

It is noted that examples of the examination target include not only a large bowel but also any other digestive tract (digestive organ) such as a large bowel, the stomach, an esophageal, and a duodenum. Examples of the endoscope in the present disclosure include a laryngendoscope, a bronchoscope, an upper digestive tube endoscope, a duodenum endoscope, a small bowel endoscope, a large bowel endoscope, a capsule endoscope, a thoracoscope, a laparoscope, a cystoscope, a cholangioscope, an arthroscope, a spinal endoscope, a blood vessel endoscope, and an epidural endoscope.

### (2) Hardware Configuration

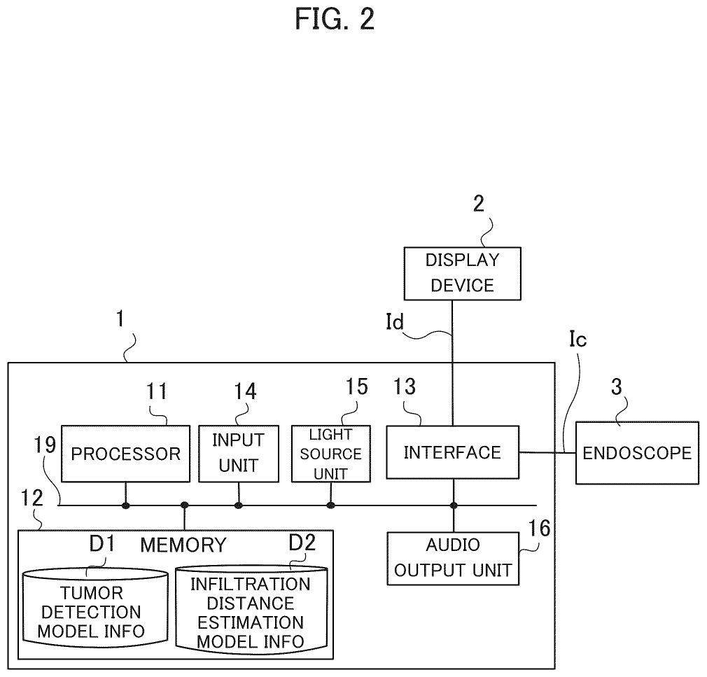

FIG. 2 shows the hardware configuration of the image processing device 1. The image processing device 1 mainly includes a processor 11, a memory 12, an interface 13, an input unit 14, a light source unit 15, and an audio output unit 16. Each of these elements is connected to each other via a data bus 19.

The processor 11 executes a predetermined process by executing a program or the like stored in the memory 12. The processor 11 is one or more processors such as a CPU (Central Processing Unit), a GPU (Graphics Processing Unit), and a TPU (Tensor Processing Unit). The processor 11 may be configured by plural processors. The processor 11 is an example of a computer.

The memory 12 is configured by a variety of volatile memories which is used as working memories, and nonvolatile memories which stores information necessary for the process to be executed by the image processing device 1, such as a RAM (Random Access Memory) and a ROM (Read Only Memory). The memory 12 may include an external storage device such as a hard disk connected to or built in to the image processing device 1, or may include a storage medium such as a removable flash memory. The memory 12 stores a program for the image processing device 1 to execute each process in the present example embodiment.

The memory 12 stores tumor detection model information D1 regarding the tumor detection model, which is a model configured to detect a tumor-containing image among inputted endoscopic images Ia, and infiltration distance estimation model information D2 regarding an infiltration distance estimation model, which is a model configured to estimate the infiltration distance of the tumor part included in the input image. The tumor detection model information D1 and infiltration distance estimation model information D2 will be described later.

The interface 13 performs an interface operation between the image processing device 1 and an external device. For example, the interface 13 supplies the display information “Id” generated by the processor 11 to the display device 2. Further, the interface 13 supplies the light generated by the light source unit 15 to the endoscope 3. The interface 13 also provides an electrical signal to the processor 11 indicative of the endoscopic image Ic supplied from the endoscope 3. The interface 13 may be a communication interface, such as a network adapter, for wired or wireless communication with the external device, or a hardware interface compliant with a USB (Universal Serial Bus), a SATA (Serial AT Attachment), or the like.

The input unit 14 generates an input signal based on the operation by the examiner. Examples of the input unit 14 include a button, a touch panel, a remote controller, and a voice input device. The light source unit 15 generates light for supplying to the tip unit 38 of the endoscope 3. The light source unit 15 may also incorporate a pump or the like for delivering water and air to be supplied to the endoscope 3. The audio output unit 16 outputs a sound under the control of the processor 11.

Next, the tumor detection model information D1 and the infiltration distance estimation model information D2 stored in the memory 12 will be described in detail.

The tumor detection model information D1 is information on the tumor detection model configured to output, once an endoscopic image is input to the model, information on whether or not the input endoscopic image Ia includes a tumor part. The tumor detection model information D1 contains the parameters required to configure the tumor detection model. The tumor detection model is, for example, a classification model configured to output, once an endoscopic image Ia is input to the model, a classification result as to the presence or absence of a tumor part in the input endoscopic image Ia. The tumor detection model may be any machine learning model (including statistical models, hereinafter the same), such as a neural network and a support vector machine. Typical examples of such neural networks include Fully Convolutional Network, SegNet, U-Net, V-Net, Feature Pyramid Network, Mask R-CNN, and DeepLab. If the tumor detection model is configured by a neural network, the tumor detection model information D1 includes parameters regarding a layer structure, a neuron structure of each layer, the number of filters and filter size in each layer, and a weight for each element of each filter, for example.

The infiltration distance estimation model information D2 is information on the infiltration distance estimation model configured to estimate, once an image obtained by photographing a part of the examination target including a tumor part is input to the model, the infiltration distance of the tumor part in the input image. The infiltration distance estimation model information D2 includes parameters required for configuring the infiltration distance estimation model. The infiltration distance estimation model is a model which has learned a relation between an image input to the infiltration distance estimation model and the infiltration distance of a tumor part of the examination target shown in the input image. The infiltration distance estimation model may be, for example, any machine learning model (including statistical models, hereinafter the same.) such as a neural network and a support vector machine. For example, if the infiltration distance estimation model is constituted by a neural network, the infiltration distance estimation model information D2 includes parameters regarding the layer structure, the neuron structure of each layer, the number of filters and the filter size in each layer, and the weight for each element of each filter.

As will be described later, the image input to the infiltration distance estimation model may be a partial image which is obtained by regularly (e.g., in a grid) cut the tumor-containing image, or may be the tumor-containing image itself. For example, in the former case, the infiltration distance estimation model outputs a numerical value indicating the estimate result of the infiltration distance at the center position of the input partial image, and in the latter case, the infiltration distance estimation model outputs an image indicating the estimate result of the infiltration distance at each pixel (or at each multiple pixel block unit or sub-pixel unit) of the entire input tumor-containing image.

In addition to the infiltration distance, the infiltration distance estimation model may be a model that further outputs the estimated result regarding the depth of each layer constituting the wall layer of the examination target shown in the image input to the infiltration distance estimation model. For example, if the examination target is a large bowel, the infiltration distance estimation model estimates the depth of each layer of the mucosal layer, lamina muscularis mucosae, submucosal layer, muscularis propria, subserosa, and serosa. If the examination target is an esophagus, the infiltration distance estimation model estimates the depth of each layer of the mucosal layer, submucosal layer, muscularis propria, and adventitia. The model for estimating the depth of each layer constituting the wall layers may be a model separated from the infiltration distance estimation model.

If the tumor detection model and the infiltration distance estimation model are learning models, the tumor detection model and the infiltration distance estimation model are trained in advance based on sets of an input image, which conforms to the input format of each model, and correct answer data indicating a correct answer to be output by each model when the input image is input to each model. The parameters of the models obtained through the training are stored in the memory 12 as the tumor detection model information D1 and the infiltration distance estimation model information D2, respectively.

### (3) Infiltration Distance Based Display Process

A description will be given of the display process based on the infiltration distance of the tumor part.

### (3-1) Outline

Schematically, upon detecting an endoscopic image Ia serving as a tumor-containing image, the image processing device 1 estimates the infiltration distance at each position of the examination target shown in the tumor-containing image, and displays an image based on the estimated infiltration distance on the display device 2. Thus, the image processing device 1 can present information on the infiltration distance of the tumor part to the examiner without requiring a detailed examination such as a bioscope. Therefore, the image processing device 1 can immediately present information, which is necessary to determine the necessity of the operation, to the examiner during the endoscopic examination.

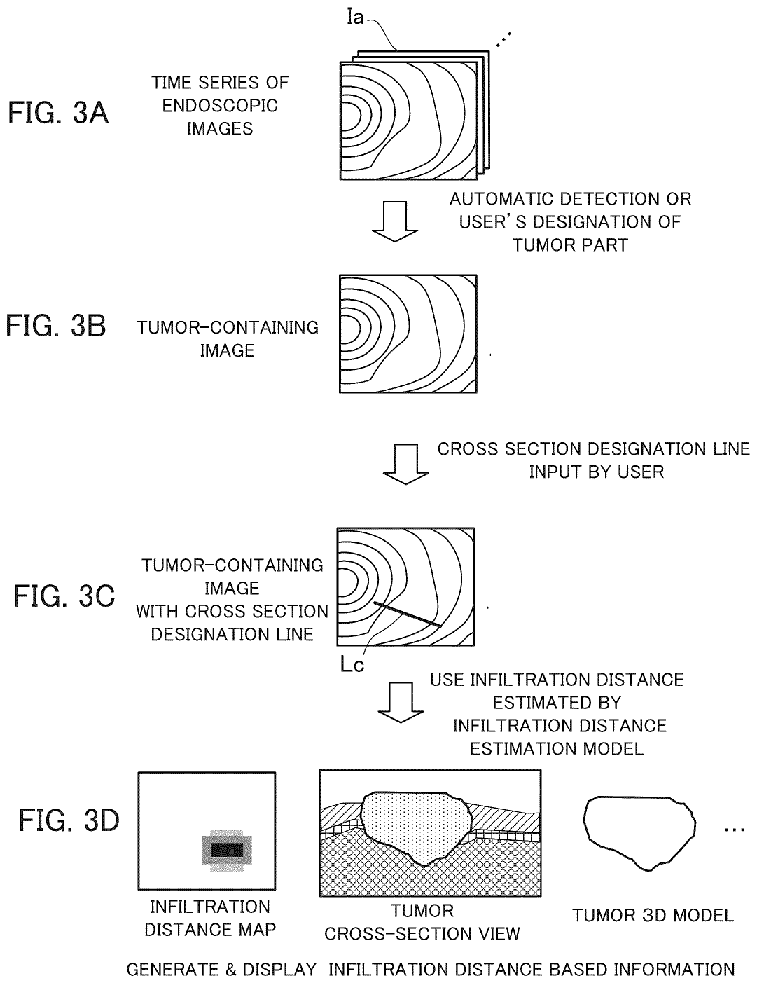

FIGS. 3A to 3D are diagrams schematically illustrating the flow of display processing based on the infiltration distance of the tumor part.

First, the image processing device 1 acquires a time series of endoscopic images Ia from the endoscope 3 as shown in FIG. 3A. Then, as shown in FIG. 3B, the image processing device 1 identifies, among the acquired endoscopic images Ia, a tumor-containing image through automatic detection of a tumor part using the tumor detection model or through user designation of a tumor part using a still image saving button of the operation unit 36, and displays the identified tumor-containing image on the display device 2.

Next, the image processing device 1 displays the tumor-containing image on the display device 2, and as shown in FIG. 3C, receives the input of the cross section designation line “Lc” for specifying the cross section of the tumor-containing part from the input unit 14 or the like. The designation of the cross section designation line Lc may be, for example, a mouse input or a touch panel input.

Then, as shown in FIG. 3D, the image processing device 1 displays on the display device 2 an image based on the infiltration distance for each position of the tumor-containing image estimated using the infiltration distance estimation model. In this case, the image processing device 1 displays an image of at least one of: a map (also referred to as “infiltration distance map”) of the infiltration distance of the examination target corresponding to the endoscopic image Ia; a cross section view (also referred to as “tumor cross section view”) of the examination target cut by the cross section designation line Lc; and a three-dimensional model (also referred to as “tumor 3D model”) representing the three-dimensional shape of the tumor part estimated based on the estimated infiltration distance. The infiltration distance map shown in FIG. 3D is a heat map where the longer the infiltration distance of the tumor part is, the darker the color becomes. The tumor 3D model may be, for example, a graphical display (e.g., wireframe display) used in CAD (Computer Aided Design).

The designation of the cross section designation line Lc may be performed after displaying the infiltration distance map. This specific example will be described later.

### (3-2) Functional Block

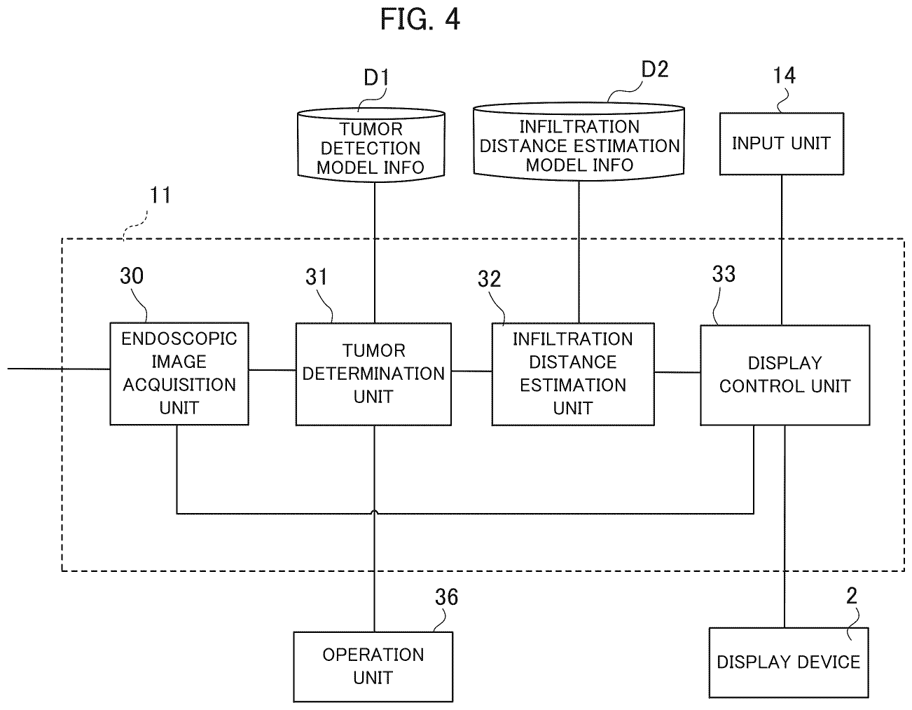

FIG. 4 is a functional block diagram of the image processing device 1 related to display processing based on the infiltration distance of the tumor part. The processor 11 of the image processing device 1 functionally includes an endoscopic image acquisition unit 30, a tumor determination unit 31, an infiltration distance estimation unit 32, and a display control unit 33. In FIG. 4, any blocks to exchange data with each other are connected to with each other by a solid line, but the combination of the blocks to exchange data with each other is not limited thereto. The same applies to the drawings of other functional blocks described below.

The endoscopic image acquisition unit 30 acquires the endoscopic image Ia taken by the endoscope 3 through the interface 13 at predetermined intervals. Then, the endoscopic image acquisition unit 30 supplies the acquired endoscopic image Ia to the tumor determination unit 31 and the display control unit 33, respectively.

The tumor determination unit 31 determines whether or not the endoscopic image Ia supplied from the endoscopic image acquisition unit 30 is a tumor-containing image. In this instance, the tumor determination unit 31 detects the endoscopic image Ia regarded as the tumor-containing image based on at least one of user input (i.e., external input) and/or an analysis result of the endoscopic image Ia. Then, upon detecting the endoscopic image Ia which serves as a tumor-containing image, the tumor determination unit 31 supplies the detected tumor-containing image to the infiltration distance estimation unit 32.

Here, the detection of the tumor-containing image based on the user input will be described. In this case, upon detecting, based on the signal supplied from the operation unit 36, that the still image saving button has been selected, the tumor determination unit 31 detects the endoscopic image Ia displayed on the display device 2 at the time of the selection as the tumor-containing image. In this instance, the tumor determination unit 31 may detect the most recent endoscopic image Ia supplied from the endoscopic image acquisition unit 30 as the tumor-containing image upon detecting that the still image saving button has been selected.

Next, the detection of the tumor-containing image based on the analysis on the endoscopic image Ia will be described. The tumor determination unit 31 inputs the endoscopic image Ia supplied from the endoscopic image acquisition unit 30 to the tumor detection model configured by referring to the tumor detection model information D1, and determines whether or not the input endoscopic image Ia is a tumor-containing image on the basis of the information outputted by the tumor detection model in response to the input of the endoscopic image Ia. For example, the tumor detection model outputs the classification result regarding the presence or absence of the tumor part in the input endoscopic image Ia, and the tumor determination unit 31 determines whether or not the input endoscopic image Ia is the tumor-containing image on the basis of the classification result.

The infiltration distance estimation unit 32 estimates, based on the infiltration distance estimation model configured by referring to the infiltration distance estimation model information D2, the infiltration distance of the tumor part of the examination target indicated by the tumor containing image supplied from the tumor determination unit 31, and supplies the estimate result to the display control unit 33. In this case, in the first example, the infiltration distance estimation unit 32 generates partial images obtained by dividing the tumor-containing image regularly (e.g., in a grid pattern). Then, the infiltration distance estimation unit 32 inputs each partial image to the infiltration distance estimation model in order, and outputs to the display control unit 33 the infiltration distance which the infiltration distance estimation model sequentially outputs for each partial image. In the second example, the infiltration distance estimation unit 32 outputs to the display control unit 33 an image which indicates the infiltration distance for each pixel of the tumor containing image, wherein the infiltration distance estimation model outputs the image in response to the input of the tumor containing image to the infiltration distance estimation model.

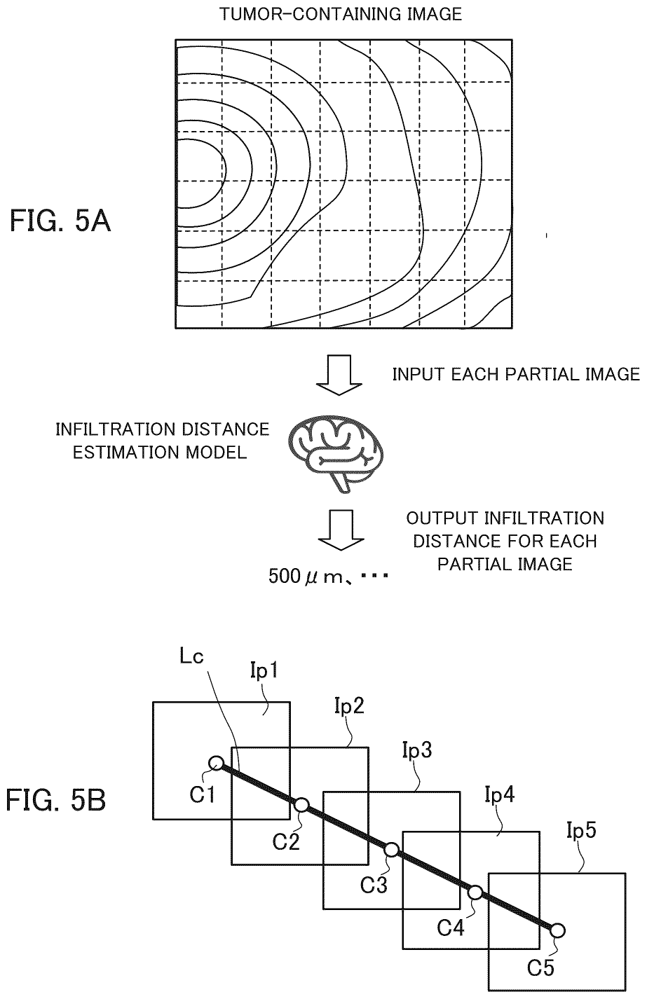

FIG. 5A shows an outline of the estimation process of the infiltration distance based on the first example described above. In the example shown in FIG. 5A, the infiltration distance estimation unit 32 generates forty-two partial images in total by dividing one tumor containing image according to horizontal seven division and vertical six division. Then, the infiltration distance estimation unit 32 inputs each partial image to the tumor detection model and thereby acquires the infiltration distance of the center position of the each partial image from the model. Thus, the infiltration distance estimation unit 32 acquires the distribution of the infiltration distance on the tumor containing image, which is necessary for generating the infiltration distance map. In some embodiments, instead of dividing the tumor containing image in a lattice shape, the infiltration distance estimation unit 32 may generate the partial images with an overlap of the partial images so that the distance of the center position of the neighboring partial images becomes shorter than the length of the partial image. This makes it possible to obtain a more detailed distribution of the infiltration distance on the tumor-containing image.

Further, if there is a setting that an infiltration distance map should not be generated and displayed, the infiltration distance estimation unit 32 may estimate the infiltration distance of only the positions along the specified cross section designation line Lc to thereby acquire the infiltration distance for displaying the tumor cross section view. FIG. 5B is a diagram showing an outline of an estimation method of the infiltration distance along the cross section designation line Lc. In the example shown in FIG. 5B, the infiltration distance estimation unit 32 sets the points C1 to C5 at equal intervals on the cross-section designation line Lc, and sets the partial images Ip1 to Ip5 which are square regions centered on the points C1 to C5, respectively. Then, the infiltration distance estimation unit 32 inputs the partial images Ip1 to Ip5 to the infiltration distance estimation model in order, and then acquires the infiltration distance at the points C1 to C5 output by the infiltration distance estimation model in order.

In some embodiments, the infiltration distance estimation unit 32 or the display control unit 33 interpolates the infiltration distance outputted by the infiltration distance estimation model through any interpolating process to thereby calculate a function or the like representing the infiltration distance at any point on the cross section designation line Lc. Thus, the infiltration distance estimation unit 32 or the display control unit 33 accurately identifies the shape of the tumor part on the cross section according to the cross section designation line Lc to thereby display tumor cross section view representing the smooth shape of the tumor part. Similarly, the infiltration distance estimation unit 32 or the display control unit 33 may generate the infiltration distance map obtained by interpolating the infiltration distance outputted by the infiltration distance estimation model in the vertical and horizontal directions through any interpolation process.

A description will be given of the display control unit 33 with reference to FIG. 4 again.

The display control unit 33 generates display information Ib based on the newest endoscopic image Ia supplied from the endoscopic image acquisition unit 30 and the estimated infiltration distance supplied from the display control unit 33. Then, the display control unit 33 supplies the generated display information Ib to the display device 2 to thereby cause the display device 2 to display an image based on the newest endoscopic image Ia and infiltration distance. Further, if the tumor determination unit 31 detects the tumor containing image, the display control unit 33 may display the latest tumor containing image in addition to or in place of the latest endoscopic image Ia on the display device 2.

In some embodiments, once the tumor determination unit 31 detects the tumor-containing image, the display control unit 33 receives the user input that specifies the cross section designation line Lc (see FIG. 3C) on the tumor-containing image or the newest endoscopic image Ia. Then, the display control unit 33 generates a tumor cross section view along the cross section designation line Lc specified by the user input and displays it on the display device 2. In this case, in some embodiments, if the infiltration distance map and the tumor cross section view should be displayed, the display control unit 33 receives the designation of the cross section designation line Lc after the display of the infiltration distance map. This enables the examiner to perform an operation of the designation of the cross section designation line Lc while confirming the position of the tumor by the infiltration distance map, and therefore suitably supports the examiner to specify the cross section designation line Lc. In some embodiments, the display control unit 33 may receive the user input for specifying the cross section designation line Lc on the infiltration distance map to thereby generate a tumor cross section view the cross section designation line Lc specified by the user input.

In some embodiments, once the tumor determination unit 31 detects the tumor-containing image, the display control unit 33 may perform the audio output control of the audio output unit 16 so as to output a warning sound or a voice guide or the like that notifies the user that the tumor part has been detected. In some embodiments, the display control unit 33 may perform the audio output control of the audio output unit 16 according to the estimation result of the infiltration distance. For example, once the tumor determination unit 31 detects the tumor containing image, the display control unit 33 may output a sound (including pitch and melody) in accordance with the infiltration distance at the center of the tumor containing image. In this case, for example, information which associates the infiltration distance at the center of the tumor-containing image with the sound to be output is stored in advance, and the display control unit 33 refers to this information and outputs a sound in accordance with the infiltration distance at the center of the tumor-containing image. Thus, the display control unit 33 may output a sound in accordance with the operation of the endoscope 3 by the examiner.

Each component of the endoscope image acquisition unit 30, the tumor determination unit 31, the infiltration distance estimation unit 32, and the display control unit 33 can be realized, for example, by the processor 11 which executes a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

### (3-3) Display Example

Next, a description will be given of the display control of the display device 2 to be executed by the display control unit 33.

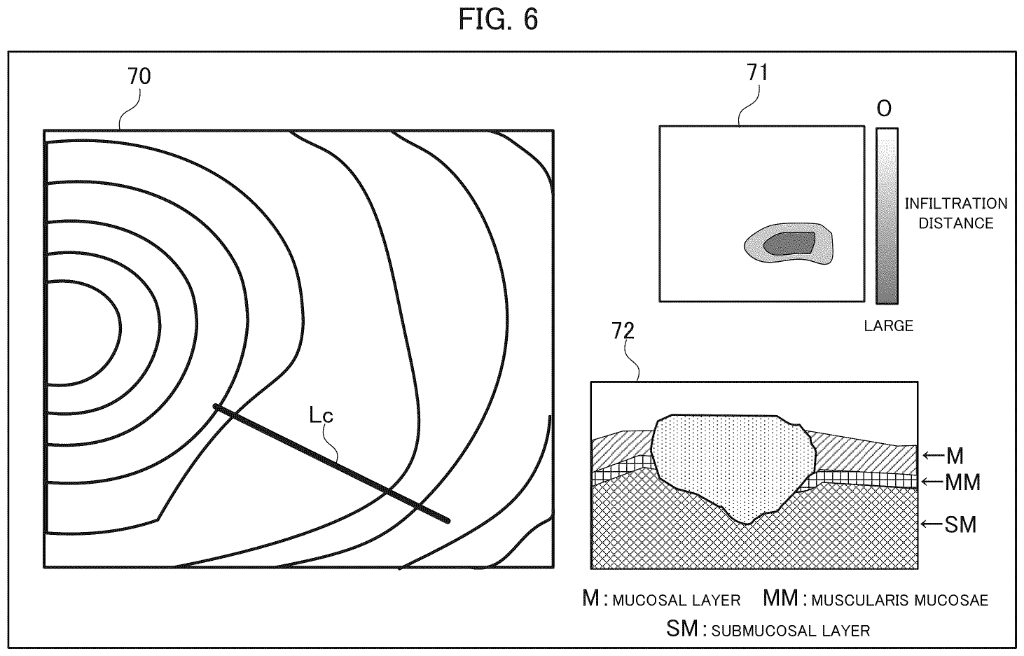

FIG. 6 shows a first display example of a display screen image displayed by the display device 2 in endoscopic examination. The display control unit 33 of the image processing device 1 transmits the display information Ib generated on the basis of the information supplied from the other processing units 30 to 32 in the processor 11 to the display device 2, so that the display screen image shown in FIG. 6 is displayed on the display device 2.

In the first display example, since the tumor determination unit 31 detects the tumor containing image, the display control unit 33 of the image processing device 1 accepts the designation of the cross section designation line Lc, and displays an image or the like on the display screen image based on the estimation result of the infiltration distance related to the detected tumor containing image. Specifically, the display control unit 33 displays an endoscopic image 70, the infiltration distance map 71, and a tumor cross section view 72 on the display screen image.

The endoscopic image 70 represents a moving image based on the latest endoscopic image Ia acquired by the endoscopic image acquisition unit 30 or a still image of the latest tumor-containing image detected by the tumor determination unit 31. Instead of the display shown in FIG. 6, the display control unit 33 may display both of the moving image based on the latest endoscopic image Ia and the still image of the latest tumor containing image detected by the tumor determination unit 31 on the display screen image. The display control unit 33 displays the cross section designation line Lc specified by the user input on the endoscopic image 70.

Further, the display control unit 33 estimates the infiltration distance of the latest tumor containing image based on the infiltration distance estimation model, and displays the infiltration distance map 71 indicating the estimate result. Here, as an example, the display control unit 33 displays the infiltration distance map 71 indicating the contour lines each of which connects the positions corresponding to the same infiltration distance among infiltration distances at predetermined intervals.

Further, the display control unit 33 displays the tumor cross section view 72 based on the infiltration distance along the cross section designation line Lc. Here, the mucosal layer (M), the lamina muscularis mucosae (MM), and the submucosal layer (SM) that constitute the wall layer of the large bowel, which is the examination target, are also clearly shown on the tumor cross section view 72.

Here, a description will be given of the specific example of the generation method of the tumor cross section view 72.

In the first example, the display control unit 33 generates the tumor cross section view 72 based on an estimate result of the depth of each wall layer of the large bowel output by the infiltration distance estimation model in response to input of a partial image or the tumor-containing image to the model, wherein the infiltration distance estimation model has been trained to estimate not only the infiltration distance but also the depth of each wall layer of the large bowel. In this instance, for each wall layer, the display control unit 33 may interpolate the estimated depth of each wall layer along the cross section designation line Lc and generate the tumor cross section view 72 based on the depth of each wall layer obtained by the interpolation. In the second example, the information indicating the standard value of the depth of each wall layer of the large bowel is stored in the memory 12, and the display control unit 33 refers to this information and generates a tumor cross section view 72 in which the depth of each wall layer is set to the above-described standard value. In the tumor cross section view 72, the bottom of the submucosal layer (SM) is drawn to coincide with the lower end of the figure.

The display control unit 33 may display, in addition to the infiltration distance map 71 and the tumor cross section view 72, or, instead of them, the tumor 3D model on the display screen image. In this case, based on the estimate result of the infiltration distance in the tumor-containing image, the display control unit 33 geometrically identifies the three-dimensional shape of the tumor part, and displays a tumor 3D model representing the identified three-dimensional shape on the display screen image.

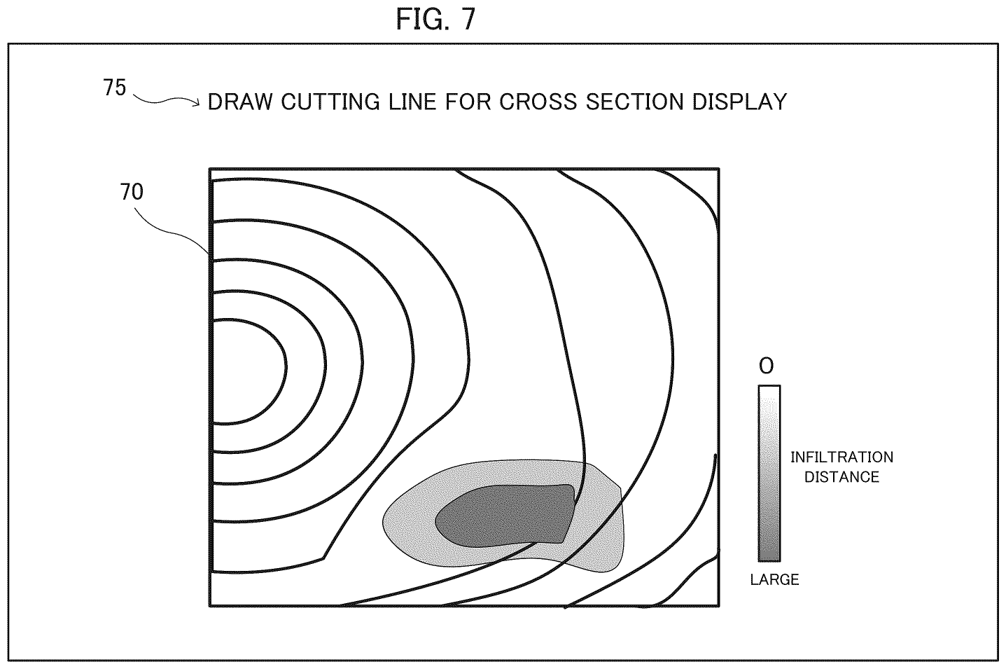

FIG. 7 shows a second display example of a display screen image displayed by the display device 2 in the endoscopic examination. The display control unit 33 of the image processing device 1 transmits the display information Ib generated on the basis of the information supplied from the other processing units 30 to 32 in the processor 11 to the display device 2, so that the display screen image shown in FIG. 7 is displayed on the display device 2.

In the second display example, once the tumor determination unit 31 detects the tumor-containing image, the display control unit 33 of the image processing device 1 generates an infiltration distance map based on the estimate result of the infiltration distance with respect to the detected tumor-containing image to display the infiltration distance map superimposed on the endoscopic image 70. Here, the infiltration distance map superimposed on the endoscopic image 70 is the same as the infiltration distance map 71 shown in FIG. 6, and it is displayed with a predetermined transmittance so that the endoscopic image 70 is visible.

Then, the display control unit 33 displays the text message 75 prompting the input of the cross section designation line Lc on the display screen image. The display control unit 33 receives the designation of the cross section designation line Lc on the basis of the operation of the input unit 14 by the examiner. Then, upon detecting the signal of the input unit 14 for specifying the cross-section designation line Lc, the display control unit 33 identifies the cross section designation line Lc to generate a tumor cross section view based on the cross section designation line Lc and display the tumor cross section view on the display screen image.

Thus, according to the second display example, the display control unit 33 can suitably accept the designation of the cross section designation line Lc for displaying the tumor cross section view. Instead of the second display example, the display control unit 33 may display the infiltration distance map and the endoscopic image 70 side by side without overlapping with each other, and receive an input to specify the cross section designation line Lc on the infiltration distance map or the endoscopic image 70.

### (3-4) Processing Flow

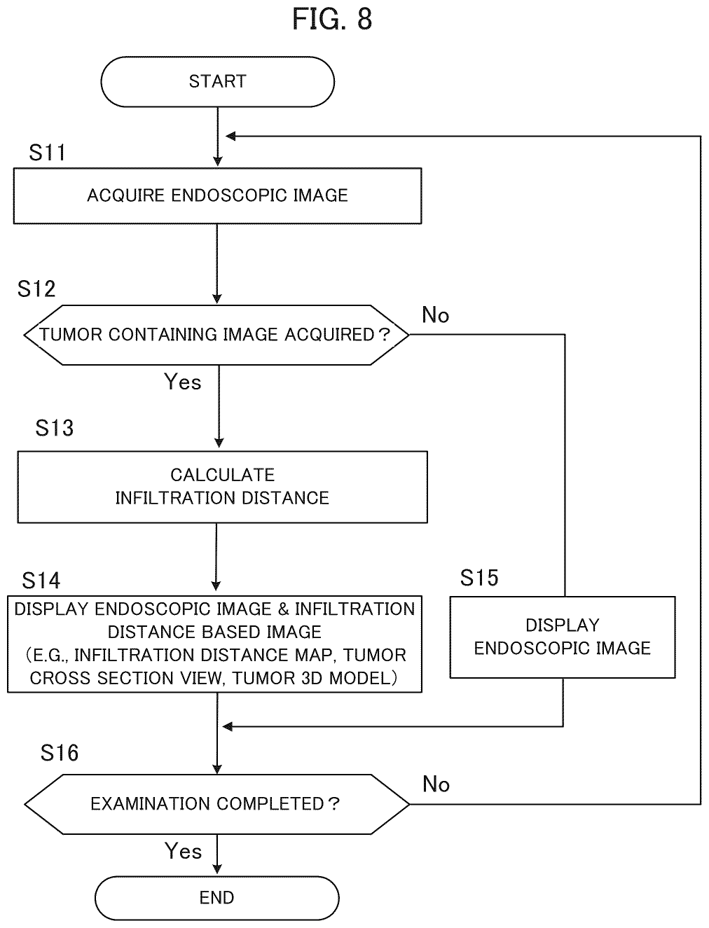

FIG. 8 is an example of a flowchart illustrating an outline of a process that is executed by the image processing device 1 during the endoscopic examination in the first example embodiment.

First, the image processing device 1 acquires the endoscopic image Ia (step S11). In this instance, the endoscopic image acquisition unit 30 of the image processing device 1 receives the endoscopic image Ia from the endoscope 3 through the interface 13.

Next, the image processing device 1 determines whether or not the endoscopic image Ia acquired at step S11 corresponds to the tumor-containing image including the tumor part (step S12). In this case, the image processing device 1 makes the above-described determination based on the information output by the tumor detection model once the endoscopic image Ia is inputted into the tumor detection model configured based on the tumor detection model information D1.

Then, upon determining that the endoscopic image Ia acquired at step S11 is a tumor-containing image (step S12; Yes), the image processing device 1 calculates the infiltration distance (step S13). In this case, the image processing device 1 acquires the infiltration distance that the infiltration distance estimation model outputs upon inputting the tumor-containing image or a partial image thereof to the infiltration distance estimation model, which is configured on the basis of the infiltration distance estimation model information D2. Then, the image processing device 1 displays on the display device 2 the endoscopic image Ia acquired at step S11 and the image based on the infiltration distance calculated at step S13 (step S14). In this instance, examples of the image based on the infiltration distance include an infiltration distance map, a tumor cross section view and a tumor 3D model. When displaying the tumor cross section view, the image processing device 1 receives a user input for specifying the cross section designation line Lc on the endoscopic image Ia. In some embodiments, the image processing device 1 may display the moving image of endoscopic images Ia and the still image of the latest tumor-containing image, respectively.

On the other hand, upon determining that the endoscopic image Ia acquired at step S11 is not a tumor-containing image (step S12; No), the image processing device 1 displays the endoscopic image Ia acquired at step S11 on the display device 2 (step S15).

Then, the image processing device 1 determines whether or not the endoscopic examination has been completed after step S14 or step S15 (step S16). For example, the image processing device 1 determines that the endoscopic examination has been completed upon detecting a predetermined input or the like to the input unit 14 or the operation unit 36. Upon determining that the endoscopic examination has been completed (step S16; Yes), the image processing device 1 ends the process of the flowchart. On the other hand, upon determining that the endoscopic examination has not been completed (step S16; No), the image processing device 1 proceeds back to the process at step S11. Then, the image processing device 1 performs processes at step S11 to step S15 on the endoscopic image Ia newly generated by the endoscope 3.

### (4) Modifications

Next, modifications suitable for the above-described example embodiment will be described. The following modifications may be applied to the example embodiments described above in any combination.

### First Modification

In displaying the infiltration distance map, the image processing device 1 may generate the infiltration distance map for a portion of the tumor-containing image, instead of generating the infiltration distance map for the entire tumor-containing image.

For example, the image processing device 1 generates and displays an infiltration distance map targeting a rectangular area (for example, the smallest rectangular area including the cross section designation line Lc) including the cross section designation line Lc specified by the examiner. In this instance, the image processing device 1 identifies the cross section designation line Lc based on a user input specifying the cross section designation line Lc, and then displays the infiltration distance map and the tumor cross section view. In this aspect, the image processing device 1 may display an infiltration distance map limited to the region of interest by the examiner.

### Second Modification

The image processing device 1 may automatically set the cross section designation line Lc instead of displaying the cross section designation line Lc specified on the basis of user input.

In this case, for example, the image processing device 1 generates an infiltration distance map for the entire tumor-containing image, and sets a predetermined length of a cross section designation line Lc passing through at least the point corresponding to the longest infiltration distance in the infiltration distance map. In another example, the image processing device 1 approximates an area where the infiltration distance is equal to or larger than a predetermined distance by an ellipse, and sets a cross section designation line Lc corresponding to the major axis of the approximate ellipse. According to this aspect, the image processing device 1 can display a tumor cross section view relating to the tumor part or the like, regardless of the input from the examiner.

### Third Modification

The image processing device 1 may process a video, which is a time series of the endoscopic images Ia generated during the endoscopic examination, after the examination.

For example, once an image to be processed is designated based on the user input by the input unit 14 at any timing after the examination, the image processing device 1 sequentially performs processing of the flowchart shown in FIG. 8 for a time-series of endoscopic images Ia constituting the video. Then, the image processing device 1 terminates the processing of the flowchart upon determining that the target video has ended at step S16. In contrasts, upon determining that the target video has not ended, it proceeds back to the process step S1 and continues the processing of the flowchart for the subsequent time series of endoscopic images Ia.

### Fourth Modification

The tumor detection model information D1 and the infiltration distance estimation model information D2 may be stored in a storage device separated from the image processing device 1.

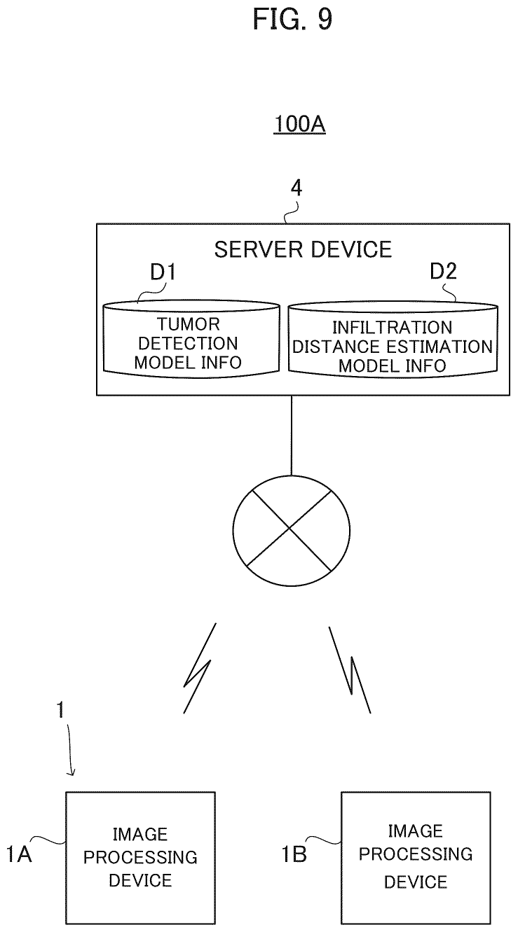

FIG. 9 is a schematic configuration diagram illustrating an endoscopic examination system 100A according to the third modification. For simplicity, the display device 2 and the endoscope 3 and the like are not shown. The endoscopic examination system 100A includes a server device 4 that stores tumor detection model information D1 and infiltration distance estimation model information D2. Further, the endoscopic examination system 100A includes a plurality of image processing devices 1 (1A, 1B, . . . ) capable of data communication with the server device 4 via a network.

In this instance, the respective image processing devices 1 refer to the tumor detection model information D1 and the infiltration distance estimation model information D2 through the network. In this case, the interface 13 of each image processing device 1 includes a communication interface such as a network adapter for performing data communication. In this configuration, the respective image processing devices 1 can suitably perform processing related to the lesion detection by referring to the tumor detection model information D1 and the infiltration distance estimation model information D2, as in the above-described example embodiment.

### Second Example Embodiment

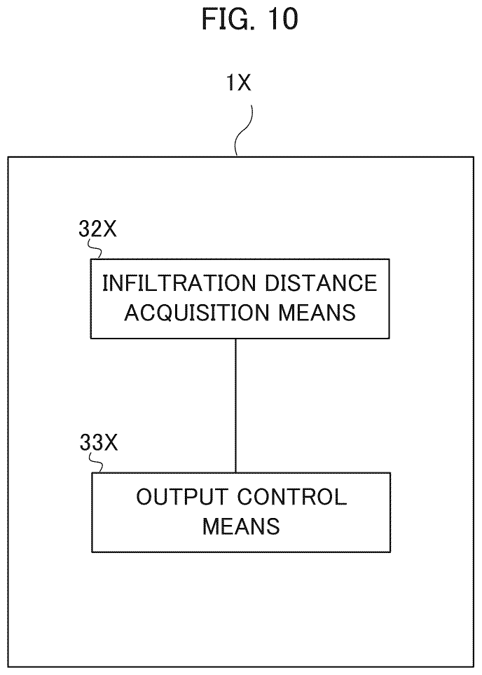

FIG. 10 is a block diagram of an image processing device 1X according to the second example embodiment. The image processing device 1X includes an infiltration distance acquisition means 32X and an output control means 33X. The image processing device 1X may be configured by a plurality of devices.

The infiltration distance acquisition means 32X is configured to acquire, based on an endoscopic image obtained by photographing an examination target by a photographing unit provided in an endoscope, an infiltration distance of a tumor part of the examination target in the endoscopic image. Examples of the infiltration distance acquisition means 32X include the infiltration distance estimation unit 32 according to the first example embodiment (including modifications, hereinafter the same). In another example, the infiltration distance acquisition means 32X may acquire the infiltration distance described above by receiving, from an external device (i.e., a device separated from the image processing device 1X) for executing a process corresponding to the infiltration distance estimation unit 32 in the first example embodiment, an estimate result of the infiltration distance of the tumor part of the examination target.

The output control means 33X is configured to output an image or sound based on the infiltration distance by an output device. The output control means 33X may be the display control unit 33 in the first example embodiment. The output device may be at least one of the display device 2 or audio output unit 16 in the first example embodiment. The output device may be incorporated in the image processing device 1X.

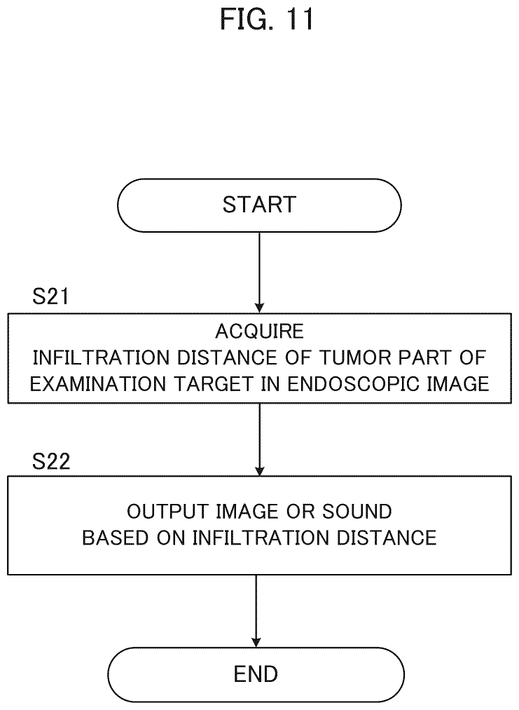

FIG. 11 is an example of a flowchart showing a processing procedure in the second example embodiment. The infiltration distance acquisition means 32X acquires, based on an endoscopic image obtained by photographing an examination target by a photographing unit provided in an endoscope, an infiltration distance of a tumor part of the examination target in the endoscopic image (step S21). The output control means 33X outputs an image or sound based on the infiltration distance by an output device (step S22).

According to the second example embodiment, the image processing device 1X can present information regarding the infiltration distance of the tumor part of the examination target included in the endoscopic image obtained by photographing the examination target.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### Supplementary Note 1

An image processing device comprising:
an infiltration distance acquisition means configured to acquire,
   based on an endoscopic image obtained by photographing an examination target by a photographing unit provided in an endoscope,
   an infiltration distance of a tumor part of the examination target in the endoscopic image; and
an output control means configured to output an image or sound based on the infiltration distance by an output device.

### Supplementary Note 2

The image processing device according to Supplementary Note 1,
wherein the output control means is configured to cause the output device to display, as the image based on the infiltration distance, a map of the infiltration distance in the endoscopic image which contains the tumor part.

### Supplementary Note 3

The image processing device according to Supplementary Note 2,
wherein the map is
   a contour map of the infiltration distance or
   a heat map of the infiltration distance other than the contour map.

### Supplementary Note 4

The image processing device according to Supplementary Note 1,
wherein the output control means is configured to cause the output device to display, as the image based on the infiltration distance, a cross section view of the examination target at the tumor part.

### Supplementary Note 5

The image processing device according to Supplementary Note 4,
wherein the cross section view includes the tumor part and a wall layer of the examination target.

### Supplementary Note 6

The image processing device according to Supplementary Note 4 or 5,
wherein the output control means is configured to cause the output device to display the cross section view cut along a line designated on the endoscopic image displayed on the output device.

### Supplementary Note 7

The image processing device of Supplementary Note 6,
wherein the output control means is configured to cause the output device to
   display a map of the infiltration distance in the endoscopic image which contains the tumor part, and
   thereafter receive an external input specifying the line.

### Supplementary Note 8

The image processing device according to Supplementary Note 1,
wherein the output control means is configured to cause the output device to display a three dimensional model of the tumor part as the image based on the infiltration distance.

### Supplementary Note 9

The image processing device according to Supplementary Note 1, further comprising
a tumor determination means configured to identify an endoscopic image which contains the tumor part among the endoscopic images captured by the photographing unit,
wherein the infiltration distance acquisition means is configured to estimate the infiltration distance based on the identified endoscopic image which contains the tumor part.

### Supplementary Note 10

The image processing device according to Supplementary Note 9,
wherein the infiltration distance acquisition means is configured to estimate the infiltration distance, based on a model to which the identified endoscopic image which contains the tumor part or a partial image of the identified endoscopic image is input, and
wherein the model is a model which has learned a relation between an input image to the model and the infiltration distance of the examination target shown in the input image.

### Supplementary Note 11

An image processing method executed by a computer, the image processing method comprising:
acquiring,
   based on an endoscopic image obtained by photographing an examination target by a photographing unit provided in an endoscope,
   an infiltration distance of a tumor part of the examination target in the endoscopic image; and
outputting an image or sound based on the infiltration distance by an output device.

### Supplementary Note 12

A storage medium storing a program executed by a computer, the program causing the computer to:
acquire,
   based on an endoscopic image obtained by photographing an examination target by a photographing unit provided in an endoscope,
   an infiltration distance of a tumor part of the examination target in the endoscopic image; and
output an image or sound based on the infiltration distance by an output device.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All patent and Non-Patent Literatures mentioned in this specification are incorporated by reference in its entirety.

### DESCRIPTION OF REFERENCE NUMERALS

1, 1A, 1B, 1X Image processing device
2 Display device
3 Endoscope
11 Processor
12 Memory
13 Interface
14 Input unit
15 Light source unit
16 Audio output unit
100, 100A Endoscopic examination system

What is claimed is:

1. An image processing device comprising:
at least one memory configured to store instructions; and
at least one processor configured to execute the instructions to:
acquire,
based on an endoscopic image obtained by photographing an examination target by an endoscope,
an infiltration distance of a tumor part of the examination target in the endoscopic image; and
output an image or sound based on the infiltration distance by an output device.

2. The image processing device according to claim 1,
wherein the at least one processor is configured to execute the instructions to cause the output device to display, as the image based on the infiltration distance, a map of the infiltration distance in the endoscopic image which contains the tumor part.

3. The image processing device according to claim 2,
wherein the map is
a contour map of the infiltration distance or
a heat map of the infiltration distance other than the contour map.

4. The image processing device according to claim 1,
wherein the at least one processor is configured to execute the instructions to cause the output device to display, as the image based on the infiltration distance, a cross section view of the examination target at the tumor part.

5. The image processing device according to claim 4,
wherein the cross section view includes the tumor part and a wall layer of the examination target.

6. The image processing device according to claim 4,
wherein the at least one processor is configured to execute the instructions to cause the output device to display the cross section view cut along a line designated on the endoscopic image displayed on the output device.

7. The image processing device of claim 6,
wherein the at least one processor is configured to execute the instructions to cause the output device to
display a map of the infiltration distance in the endoscopic image which contains the tumor part, and
thereafter receive an external input specifying the line.

8. The image processing device according to claim 1,
wherein the at least one processor is configured to execute the instructions to cause the output device to display a three dimensional model of the tumor part as the image based on the infiltration distance.

9. The image processing device according to claim 1,
wherein the at least one processor is configured to further execute the instructions to identify an endoscopic image which contains the tumor part among the endoscopic images captured by the endoscope, and
wherein the at least one processor is configured to execute the instructions to estimate the infiltration distance based on the identified endoscopic image which contains the tumor part.

10. The image processing device according to claim 9,
wherein the at least one processor is configured to execute the instructions to estimate the infiltration distance, based on a model to which the identified endoscopic image which contains the tumor part or a partial image of the identified endoscopic image is input, and
wherein the model is a model which has performed a machine learning of a relation between an input image to the model and the infiltration distance of the examination target shown in the input image.

11. An image processing method executed by a computer, the image processing method comprising:
acquiring,
based on an endoscopic image obtained by photographing an examination target by an endoscope,
an infiltration distance of a tumor part of the examination target in the endoscopic image; and
outputting an image or sound based on the infiltration distance by an output device.

12. A non-transitory computer readable storage medium storing a program executed by a computer, the program causing the computer to:
acquire,
based on an endoscopic image obtained by photographing an examination target by an endoscope,
an infiltration distance of a tumor part of the examination target in the endoscopic image; and
output an image or sound based on the infiltration distance by an output device.
